(19) United States
(12) Patent Application Publication
(10) Pub. No.: US 2009/0155324 A1
(43) Pub. Date: Jun. 18, 2009

(54) Skin Treatment System

(76) Inventors: Gina Achin, Cohasset, MA (US); Erla Esposito, Cohasset, MA (US)

Correspondence Address: HOLLAND & KNIGHT LLP, 10 ST. JAMES AVENUE, 11th Floor, BOSTON, MA, 02116-3889, US

(21) Appl. No.: 11/956,837

(22) Filed: Dec. 14, 2007

(51) Int. Cl. A61F 13/00; A61K 45/00; A61P 17/00

(52) U.S. Cl. 424/402; 514/789; 602/48; 604/308; 604/304

(57) ABSTRACT

A skin treatment system includes a flexible panel having an outer surface and an inner surface. The flexible panel may be configured to surround at least a portion of a user's appendage. The skin treatment system may also include at least one skin treatment insert having an inner surface and an outer surface. The flexible panel may be further configured to releasably engage the skin treatment insert. The skin treatment insert may be configured to receive a topical skin treatment. The inner surface of the skin treatment insert may be configured to apply the topical skin treatment to at least a portion of a surface of the user's appendage.

### FIELD OF THE INVENTION

The present invention relates to skin treatment, and more specifically to a skin treatment system that enables continuous skin treatment.

### BACKGROUND

Many lotions, creams, ointments and other skin treatments exist for topically treating various skin ailments, such as dry skin, eczema and rashes. Many of these skin treatments may require frequent application and may work best when frequently applied. While each application of such skin treatments may provide temporary relief, such relief may diminish after the skin treatment has absorbed into the skin. Further, skin treatments may be less effective if wiped away due to contact with clothing or bed linens. Thus, there exists a need for a system that enables continuous application of a skin treatment.

### SUMMARY

According to an implementation, a skin treatment system may include a flexible panel having an outer surface and an inner surface. The flexible panel may be configured to surround at least a portion of an appendage of a user. The skin treatment system may also include at least one skin treatment insert having an inner surface and an outer surface. The flexible panel may be further configured to releasably engage the at least one skin treatment insert. The at least one skin treatment insert may be configured to receive a topical skin treatment. The inner surface of the at least one skin treatment insert may be configured to apply the topical skin treatment to at least a portion of a surface of the appendage.

One or more of the following features may be included. At least one inner panel may be operably connected to the flexible panel, thereby forming at least one pocket configured to receive the at least one skin treatment insert. The at least one inner panel may be configured to enable application of the topical skin treatment to at least a portion of a surface of the appendage.

At least one removable fastener may be configured to secure the flexible panel to at least a portion of an appendage of a user. The at least one removable fastener may be at least one of a hook and loop fastener, a button, a snap, a zipper or a two-piece buckle. The at least a portion of an appendage of a user may be one of a foot and an elbow. The topical skin treatment may be at least one of moisturizing lotion, moisturizing cream, or topical medication.

The at least one skin treatment insert may be disposable. The at least one skin treatment insert may be refillable. The flexible panel may be made from a non-breathable material. The at least one skin treatment insert may be configured to prevent communication of the topical skin treatment to the flexible panel.

The skin treatment system can be implemented to realize one or more of the following advantages. For example, the skin treatment system may enable a wearer to moisturize or otherwise treat her skin while sleeping, without having the topical skin treatment rub off on bedding. Similarly, a wearer may treat his skin during the day without having the topical skin treatment rub off on clothing. Additionally, the skin treatment system enables continuous application of a topical skin treatment, enabling a wearer to avoid multiple applications over the course of a day.

Other features and advantages will become apparent from the description, the drawings and the claims. The details of one or more implementations are set forth in the accompanying drawings and the description below.

### BRIEF DESCRIPTION OF THE DRAWINGS

FIG. 1 is a front view of an exemplary skin treatment system according to one embodiment.

FIG. 2 is a side view of an exemplary skin treatment system applied to an elbow of a wearer.

FIG. 3 is a side view of an exemplary skin treatment system applied to a foot of a wearer.

FIG. 4 is a front view of an exemplary skin treatment insert.

FIG. 5 is a front view of an exemplary skin treatment system according to another embodiment.

FIG. 6 is a front view of an exemplary skin treatment system according to another embodiment.

Like reference numbers and designations in the various drawings indicate like elements.

### DETAILED DESCRIPTION

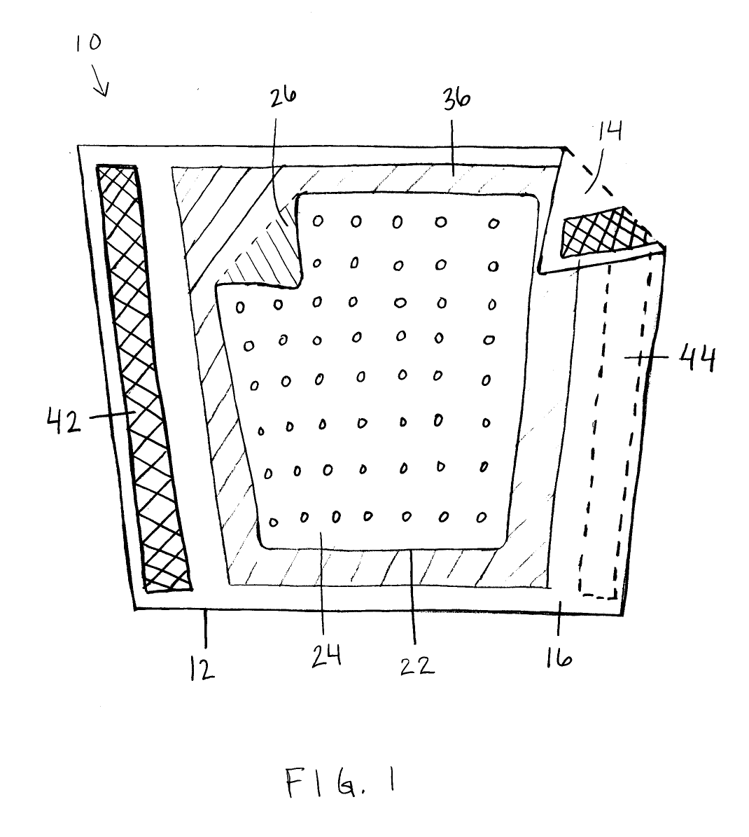

As seen in FIG. 1, an exemplary skin treatment system 10 may include a flexible panel 12 having an outer surface 14 and an inner surface 16. The flexible panel 12 may be configured to surround at least a portion of an appendage of a user. The flexible panel 12 may be constructed from various flexible materials. For example, the flexible panel 12 may be made of a natural or synthetic fabric such as cotton, polyester, spandex, lycra or any combination thereof. The shape and size of the flexible panel 12 may vary depending upon the type of appendage to which it is to be applied, as well as upon the size of the wearer. In some embodiments, the flexible panel 12 may be made from a non-breathable material. For example, the flexible panel 12 may include a layer of plastic or other non-permeable and/or non-breathable material to prevent transmission of a skin treatment through the flexible panel 12.

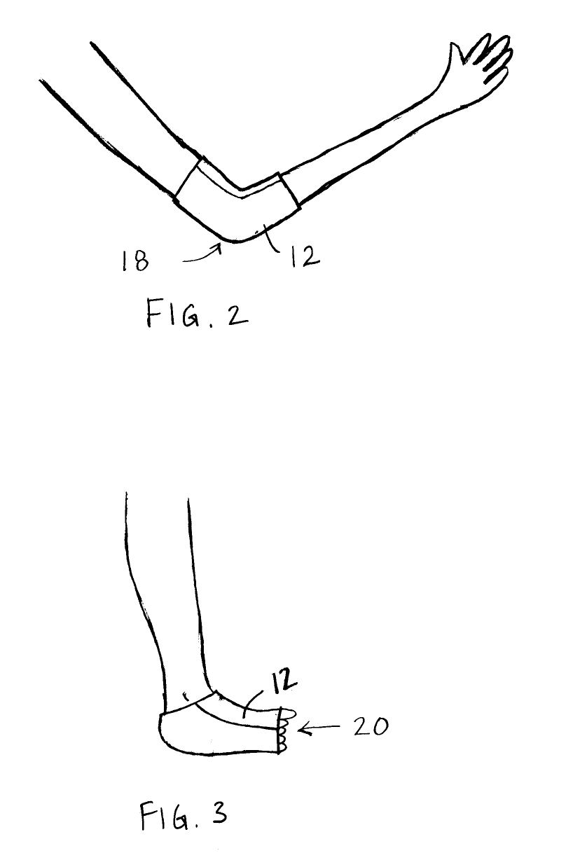

Referring also to FIGS. 2 and 3, the at least a portion of an appendage of a user may be one of an elbow 18 and a foot 20, or any other appendage requiring a topical skin treatment. For example, a flexible panel 12 for application to an elbow 18 may be rectangular or trapezoidal in shape, while a flexible panel 12 for application to a foot 20 may be configured to wrap at least partially around the ankle of a wearer in addition to the foot 20 to better secure the flexible panel 12 to the foot 20.

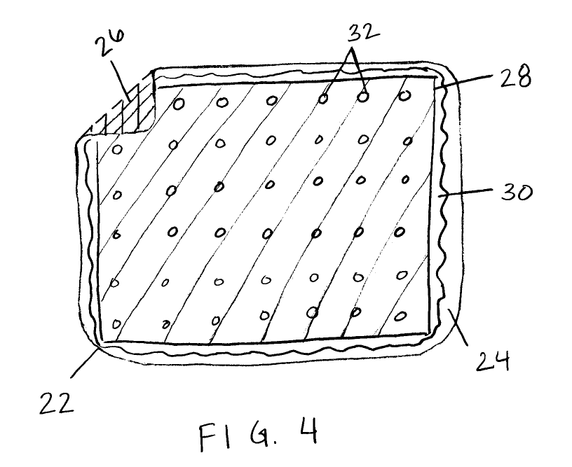

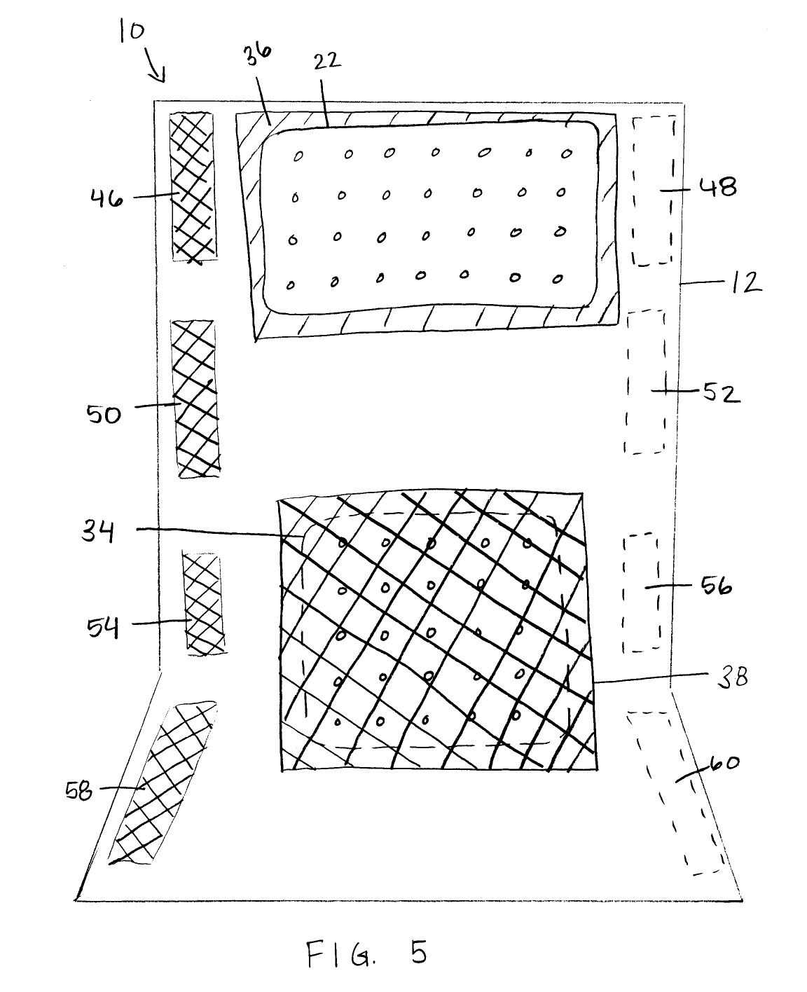

Referring to FIGS. 1 and 4, the skin treatment system 10 may also include at least one skin treatment insert 22 including an inner surface 24 and an outer surface 26. The skin treatment insert 22 may be a pouch or pocket. The skin treatment insert 22 may be constructed from a variety of materials. The inner surface 24 and the outer surface 26 may be made from the same material. Alternatively, the inner surface 24 and the outer surface 26 may be made from different materials. For example, the outer surface 26 may be made from a non-permeable material such as plastic and the inner surface 24 may be made from a permeable material. In embodiments where the inner surface 24 is permeable, a peal-away non-permeable layer 28 may be placed over the inner surface 24 until it is ready for application.

The at least one skin treatment insert 22 may be configured to receive a topical skin treatment 30. For example, skin treatment insert 22 may be a pouch or pocket configured to contain a topical skin treatment 30 such as moisturizing lotion, cream, ointment or other topical treatments and medications. The inner surface 24 of skin treatment insert 22 may be configured to apply the topical skin treatment 30 to the at least a portion of a surface of the appendage. Continuing with the above example, the inner surface 24 of skin treatment insert 22 may be made from a permeable material that allows the topical skin treatment 30 to be communicated from skin treatment insert 22 to a wearer's skin. Alternatively, the inner surface 24 may include openings 32 configured to allow the topical skin treatment 30 to pass through to the skin of a wearer.

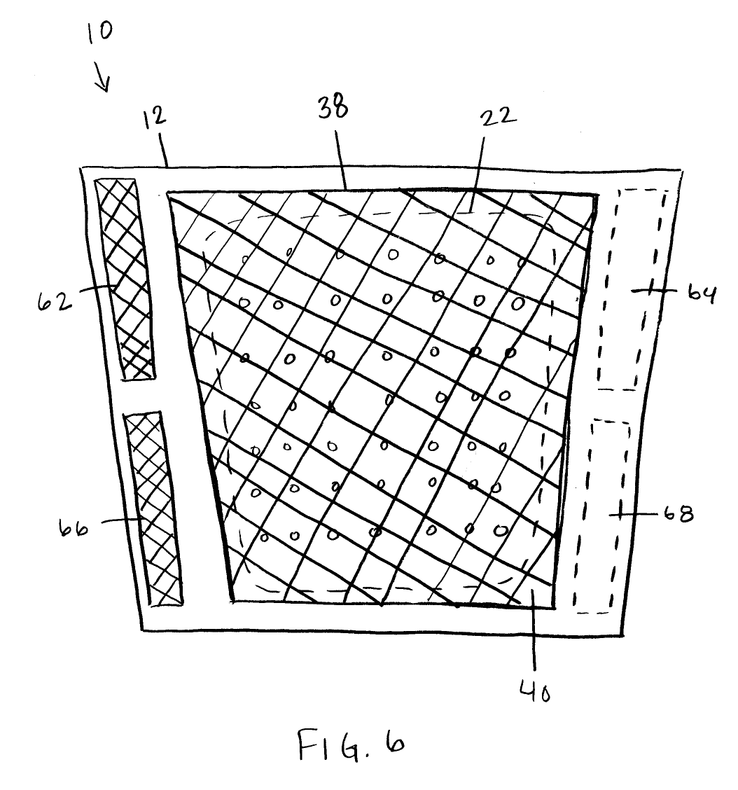

Referring also to FIG. 5, more than one skin treatment insert 22, 34 may be included in skin treatment system 10 if multiple regions on the appendage of the wearer require application of a topical skin treatment 30. For example, a flexible panel 12 configured for application to a foot may be configured to releasably engage skin treatment insert 22 for the ball of the foot and skin treatment insert 34 for the heel.

The skin treatment inserts 22, 34 may be configured to prevent communication of the topical skin treatment 30 to the flexible panel 12. Continuing with the above example, the outer surface 26 of the skin treatment insert 22 may be constructed from or may include a layer of plastic or other non-permeable and/or non-breathable material to prevent transmission of the topical skin treatment 30 through the outer surface 26 of the skin treatment insert 22 to the flexible panel 12.

The topical skin treatment 30 may be, for example, a least one of moisturizing lotion, moisturizing cream, and topical medication. For example, a moisturizing lotion and/or moisturizing cream may be used to treat eczema or dry skin. A topical analgesic may be used to treat a burn. A topical antihistamine or hydrocortisone may be used to treat an allergy-related rash or other dermatitis.

The flexible panel 12 may be further configured to releasably engage skin treatment insert 22. For example, the inner surface 16 of the flexible panel 12 may include an adhesive layer 36, a hook and loop type fastener (not shown) or other mechanism for releasably engaging the skin treatment insert 22. Similarly, the outer surface 26 of the skin treatment insert 22 may include an adhesive layer (not shown), a hook and loop type fastener (not shown) or other mechanism for releasably engaging the flexible panel 12.

Referring also to FIG. 6, at least one inner panel 38 may be operably connected to the flexible panel 12, thereby forming at least one pocket 40 configured to receive the skin treatment insert or inserts 22, 34. The inner panel 38 may be permanently or removably attached to the flexible panel 12. For example, the inner panel 38 may be configured to snap, zip, hook and loop fasten or otherwise removably attach to the flexible panel 12. Alternatively, the inner panel 38 may be stitched, glued or otherwise permanently attached to the flexible panel 12. Whether permanently or removably attached to the flexible panel 12, the inner panel 38 may include an opening for insertion of at least one skin treatment insert 22, 34.

The inner panel 38 may be configured to allow application of the topical skin treatment 30 to the surface of the appendage. For example, the inner panel 38 may be configured to prevent adhesion of the skin treatment insert 22, 34 to the skin of a wearer. The inner panel 38 may be constructed from a permeable material such as a natural or synthetic mesh. The topical skin treatment 30 from the skin treatment insert 22, 34 may pass through the inner panel 38 to the skin of a wearer.

At least one removable fastener 42 may be configured to secure the flexible panel 12 to the appendage of a user. For example, the flexible panel 12 may be configured to releasably engage itself when wrapped around an appendage of a wearer. The removable fastener may be disposed adjacent the edges of the flexible panel such that the edges of the flexible panel 12 may be removably attached when brought into or near contact. The at least one removable fastener may be at least one of a hook and loop fastener, a button, a snap, a zipper or a two-piece buckle. For example, in FIG. 1 a first and second half 42, 44 of a removable fastener such as a hook and loop fastener may be utilized to secure the flexible panel 12 around an appendage of a wearer. Similarly, in FIGS. 5 and 6, multiple removable fasteners may be utilized (e.g., first and second halves of removable fasteners 46, 48, 50, 52, 54, 56, 58, 60, 62, 64, 66, 68).

The at least one skin treatment insert 22, 34 may be disposable. For example, the skin treatment insert 22, 34 may be pre-filled with topical skin treatment 30 for one use only and then thrown away after the topical skin treatment 30 has been expended. Alternatively, the skin treatment insert 22, 34 may be refillable. For example, the skin treatment insert 22, 34 may be configured for multiple uses and may include a valve or other opening (not shown) enabling the skin treatment insert 22, 34 to be refilled with topical skin treatment 30.

It is to be understood that the foregoing description is intended to illustrate and not to limit the scope of the invention, which is defined by the scope of the appended claims. Other embodiments are within the scope of the following claims. For example, while the at least one skin treatment insert 22, 34 has been described as removable from the flexible panel 12, the at least one skin treatment insert may be permanently disposed in or on the flexible panel and may be refillable. Further, while hook and loop type fasteners have been illustrated as the removable fasteners, snaps, buttons, zippers and other fastening mechanisms may be utilized to secure the flexible panel 12 to the at least a portion of an appendage of a wearer. Similarly, while openings in the inner surface 24 of the skin treatment insert 22, 34 have been illustrated as allowing transmission of the topical skin treatment 30 from the skin treatment insert 22, 34 to the skin of a wearer, a permeable material may be utilized for the same purpose.

What is claimed is:

1. A skin treatment system comprising:
a flexible panel including an outer surface and an inner surface, the flexible panel configured to surround at least a portion of an appendage of a user; and
at least one skin treatment insert including an inner surface and an outer surface, wherein the flexible panel is further configured to releasably engage the at least one skin treatment insert, the at least one skin treatment insert configured to receive a topical skin treatment, wherein the inner surface of the at least one skin treatment insert is configured to apply the topical skin treatment to the at least a portion of a surface of the appendage.

2. The skin treatment system of claim 1 further comprising:
at least one inner panel operably connected to the flexible panel, thereby forming at least one pocket configured to receive the at least one skin treatment insert, the at least one inner panel configured to enable application of the topical skin treatment to the at least a portion of a surface of the appendage.

3. The skin treatment system of claim 1 further comprising at least one removable fastener configured to secure the flexible panel to the at least a portion of an appendage of a user.

4. The skin treatment system of claim 1 wherein the at least a portion of an appendage of a user is one of a foot and an elbow.

5. The skin treatment system of claim 3 wherein the at least one removable fastener is at least one of a hook and loop fastener, a button, a snap, a zipper and a two-piece buckle.

6. The skin treatment system of claim 1 wherein the topical skin treatment is selected from the group consisting of moisturizing lotion, moisturizing cream, and topical medication.

7. The skin treatment system of claim 1 wherein the at least one skin treatment insert is disposable.

8. The skin treatment system of claim 1 wherein the at least one skin treatment insert is refillable.

9. The skin treatment system of claim 1 wherein the flexible panel is made from a non-breathable material.

10. The skin treatment system of claim 1 wherein the at least one skin treatment insert is configured to prevent communication of the topical skin treatment to the flexible panel.
